(19) United States
(12) Patent Application Publication
(10) Pub. No.: US 2010/0105082 A1
(43) Pub. Date: Apr. 29, 2010

(54) RAPID DETECTION NANOSENSORS FOR BIOLOGICAL PATHOGENS

(75) Inventors: Dinakar RAMADURAI, Chicago, IL (US); James W. GARLAND, Aurora, IL (US); Sivalingam SIVANANTHAN, Naperville, IL (US)

Correspondence Address: Momkus McCluskey, LLC, 1001 Warrenville Road, Suite 500, Lisle, IL, 60532, US

(73) Assignee: EPIR TECHNOLOGIES, INC., Bolingbrook, IL (US)

(21) Appl. No.: 12/259,919

(22) Filed: Oct. 28, 2008

(51) Int. Cl. G01N 33/569; G01N 33/53; C12M 1/00

(52) U.S. Cl. 435/7.37; 435/7.2; 435/7.32; 435/287.2

(57) ABSTRACT

An assay test solution, a method for using, and an apparatus for the rapid detection of multiple pathogens using a FRET-based phenomenon. A volume of fluid, possibly containing pathogens, is passed through an intake and combined with an assay solution of quantum dot/antibody-antigen/quencher complexes that dissociate and recombine with the pathogens into quantum dot/antibody-pathogen complexes. The quantum dot/antibody-antigen/quencher and quantum dot/antibody-pathogen complexes are captured on a detection filter which is illuminated by a light source. The quantum dot/antibody-pathogen complexes, but not the quantum dot/antibody-antigen/quencher complexes, fluoresce when excited by the light from the light source and the fluorescence is picked up by a photodetector, indicating the presence of the pathogens.

### STATEMENT AS TO RIGHTS IN INVENTIONS MADE UNDER FEDERALLY-SPONSORED RESEARCH AND DEVELOPMENT

This invention was made with Government support under Contract No. W9132T-06-C-0032 awarded by the United States Army. The government has certain rights in this invention.

### BACKGROUND OF THE INVENTION

Political and social upheaval over the last decade combined with the dangers of chemical agents and biological pathogens have given the United States and other countries around the world an increasing cause for concern about the use of these agents against both citizens and military personnel. As a result, these concerns about public health and safety have fostered research into security systems that can detect these agents quickly and effectively.

In addition to security applications, increasing limitations on the emissions of chemicals and biological agents to the environment require quick and accurate detection as a part of monitoring and compliance programs. Thus, there is a need for quick and accurate testing of fluids for chemicals and pathogens in both the commercial and municipal areas as well.

Unfortunately, many of the systems currently in use are prone to erroneous positive results, or “false positives,” and often take too long to receive satisfactory results. These current systems are, therefore, inadequate for use in on-the-spot analysis in areas such as airports, post offices, office buildings, military installations, water treatment plants, HVAC systems, or anywhere the rapid and reliable detection of chemical or biological agents is needed.

Some of these current technologies involve the use of fluorescent resonance energy transfer (hereinafter referred to as “FRET”) technology. FRET technology uses reactions between fluorescent dyes or quantum dots (hereinafter referred to as “QDs”) and quencher molecules, which absorb the light emitted from the dyes or QDs. In the FRET process, quantum dots are subjected to light and respond by emitting a discrete wavelength of light. The associated quencher molecules then absorb the light emitted by the fluorescent dyes or QDs.

Quantum dots have been shown to have superior brightness characteristics and more discrete wavelengths when emitting light than fluorescent dyes and have been used in a variety of applications, including the imaging of cells and cell structures, the labeling of genetic markers, the tracking of glycine receptors, and chemical sensors. For example, the advantages of using semiconductor QDs as fluorescent biotags are disclosed by Wu et al., Immunofluoroscent Labeling of Cancer Marker Her2 and Other Cellular Targets with Semiconductor Quantum Dots, Nature Biotechnol., 2003, 21, 41-46. Further, Jaiswal et al., Long Term Multiple Color Imaging of Live Cells Using Quantum Dot Bioconjugates, Nature Biotechnol., 2003, 21, 47-51 have recently demonstrated that ZnS-coated CdSe QDs may be used in multiple imaging of structures in living cells. Quantum dots have been used as a chemical sensor for the detection of trinitrotoluene (TNT) by Medintz et al., Self-Assembled Nanoscale Biosensors based on QD FRET Donors, Nature Materials, 2003, 21, 630-638. However, a need still exists for a detection system that rapidly detects pathogens in a fluid (such as air or water) while having a zero or near zero incidence of false positives.

### SUMMARY OF THE INVENTION

This invention provides for biosensors for the rapid and highly sensitive and specific detection of biological pathogens through the use of FRET. A QD is bonded to an antibody conjugated with a deactivated antigen in turn bonded to one or more quencher molecules (hereinafter, QD/antibody-antigen/quencher complexes). When combined with the use of filters, the combination of quenchers and the QDs gives an increased signal to noise ratio resulting in zero or near zero false positives. As long as the QD is associated with a quencher, the QD will not exhibit detectable fluorescence.

In a method according to one aspect of the invention, pathogens are detected by formulating an assay test solution containing at least one QD/antibody-antigen/quencher complex and introducing a volume of fluid, possibly containing pathogens, into a vessel with the assay test solution to form a test sample solution. The test sample solution is then held for a predetermined period of time to permit the dissociation of a portion of the quantum dot/antibody-antigen/quencher complexes and the binding with the pathogens (if any) from the volume of fluid, to form QD/antibody-pathogen complexes. The test sample solution then is passed through one or more detection filters that traps the QD/antibody-antigen/quencher complexes and the QD/antibody-pathogen complexes while the remainder of the fluid containing any unbound QDs and QD/antibody complexes passes through. The detection filter(s) is illuminated with a light source, causing the QDs on the QD/antibody-pathogen complexes to emit a predetermined frequency of light. A photodetector detects the light from each type of QD/antibody-pathogen complex, indicating the presence of that type of pathogen. The intensity of each type of fluorescence can be calibrated to measure the concentration of the corresponding type of pathogen in the sample solution. The QD/antibody-antigen/quencher complexes do not fluoresce since the quencher molecule(s) are still attached.

Preferably, the assay test solution includes at least a kind of second QD/antibody-antigen/quencher complex for the detection of a second kind of pathogen, where the second inactivated antigen and second QD are different from the first inactivated antigen and first QD. The first and second QDs are chosen to emit discernibly different wavelengths. Similarly, further QD/antibody-antigen/quencher complexes can be devised which detect further kinds of pathogens, and emit other, discernibly different wavelengths of fluorescent light when they do.

The apparatus according to another aspect of the invention detects at least one pathogen through the use of FRET. The apparatus includes an intake, a means for drawing in a volume of fluid into the intake, a vessel or vessels containing assay test solution including at least one kind of QD/antibody-antigen/quencher complex, a conduit coupling the intake to the vessel, one or more detection filters, a means for flowing a portion of the test sample solution through the detection filter(s), a light source, and a photodetector which will detect the presence on the detection filter(s) of fluorescing QDs that are bound to pathogens. In one embodiment, there can be a plurality of detection filters with decreasing pore sizes.

Preferably, the photodetector discriminates between at least two wavelengths, so that more than one pathogen can be detected.

In another aspect of the invention, the invention provides an assay test solution for detecting one or more pathogens through the use of FRET including at least one QD bound to an antibody conjugated with at least one inactivated target antigen bound to one or more quencher molecules.

Preferably, the QDs used have a Group II-VI semiconductor core. More preferably, the QD includes a CdSe—ZnS core-shell nanocrystal.

One advantage of the present invention is that it provides for the detection of pathogens in the order of minutes, rather than hours, and provides the ability to detect multiple pathogens simultaneously. Additionally, since QDs emit light with a higher intensity than fluorescent dyes, the invention gives high signal strength and near zero background noise which gives increased sensitivity over the prior art.

### BRIEF DESCRIPTION OF THE DRAWINGS

Further aspects of the invention and their advantages can be discerned in the following detailed description, in which like characters denote like parts and in which:

FIG. 1A-1C are schematic sequential diagrams illustrating a general process of dissociation of the QD/antibody-antigen/quencher complex and resulting bonding to the pathogen;

FIG. 2 is a schematic diagram illustrating the binding reaction between QDs and antibodies;

FIG. 3 is a schematic diagram showing a filtering mechanism used for the FRET-based multiplexing scheme;

FIG. 4 is a graph showing photoluminescence (PL) measurement from a solution containing QD/antibody-antigen/quencher complexes, specifically E. coli-605 nm QDs labeled with BHQ-2 quenchers, B. cereus-565 nm QDs labeled with BHQ-2 quenchers, and MS-2-525 nm QDs labeled with BHQ-2 quenchers, a statistical fraction of which are dissociated, along with some unconjugated QDs, before and after the introduction of E. coli, B. cereus and MS-2 unlabeled pathogens;

FIG. 5A is a fluorescence image of BHQ-2 labeled E. coli 0157:H7 tagged to 605 nm QD/antibody conjugates, BHQ-2 labeled B. cereus tagged to 565 nm QD/antibody conjugates and BHQ-2 labeled MS-2 tagged to 525 nm QD/antibody conjugates, all trapped on a filter that allows unconjugated QDs and dissociated QD complexes to pass through, before the introduction of unlabeled pathogens;

FIG. 5B is a fluorescence image of BHQ-2 labeled E. coli 0157:H7 tagged to 605 nm QD/antibody conjugates, BHQ-2 labeled B. cereus tagged to 565 nm QD/antibody conjugates and BHQ-2 labeled MS-2 tagged to 525 nm QD/antibody conjugates all trapped on a filter that allows unconjugated QDs and dissociated QD complexes to pass through, after the introduction of E. coli, B. cereus and MS-2 unlabeled pathogens; and

FIG. 6 is a schematic isometric view of exemplary detection apparatus according to the invention, showing its internal structure.

### DETAILED DESCRIPTION

This invention provides a method and apparatus for the simultaneous rapid detection of several types of biological antigens. The FRET scheme used involves QDs conjugated to antibodies specific to a quencher-labeled deactivated antigen. In such complexes, the quenchers inhibit the fluorescence of the QDs when conjugated with them, providing an essentially zero noise background to the initial detection process. As used in the Specification, the following terms have the following definitions:

The term “antigen” means any chemical or biological agent, substance, or organism that provokes an immune system to produce an antibody or antibodies.

The term “inactivated antigen” means an antigen that has been reduced in potency or effectiveness.

The term “antibody” means any entity that is produced by an immune system in response to an antigen.

The term “quantum dot” or “QD” means a semiconducting nanocrystal that emits one or more discrete frequencies of light when stimulated by a light source. Additionally, when present as a part of a QD/antibody-antigen/quencher or QD/antibody-pathogen complex, it should be understood that the QD has been functionalized with an organic layer.

The term “quencher” means any inorganic or organic compound that can attach to an antigen, has strong absorbance characteristics over at least the range of wavelengths emitted by the QDs used in the invention, and has no native fluorescence of its own.

The term “photodetector” means any device capable of detecting one or more discrete frequencies of light emitted from the QDs.

The term “volume of fluid” means any defined or continuous amount fluid drawn in through the intake, particularly where the fluid is air or water.

The term “QD/antibody-antigen/quencher complex” means a QD bonded to an antibody conjugated with a deactivated antigen bonded to at least one quencher molecule.

The antigens of the present invention cover a broad range of chemical and biological agents and are intended to encompass anything that provokes a response from an immune system. Chemical agents include but are not limited to regulated and unregulated industrial chemicals, toxins, and any other chemical that provokes an immune system response. Biological agents include, but are not limited to, microscopic single-cell prokaryotes and eukaryotes including bacteria, protozoa and algae; multicellular organisms, cells from such multicellular organisms (e.g. human blood cells), spores, viruses and biological toxins. While no specific size limitation is intended, the invention can be used to detect small (approximately 30 nanometers for the MS2-virus), medium (approximately 1 micron for Bacillus cereus), and large (approximately 3 microns for Escherichia coli 0157:H7) pathogens.

The quenchers used in the present invention should at least absorb light in a range including the emission wavelengths of the employed QDs and should have no natural fluorescence of their own. Preferably the quenchers absorb a broad spectrum of light. They may be organic or inorganic molecules bound to the inactivated antigens or freely available in solution. In practice, quencher molecules located within the Frster radius of a QD (the distance at which energy transfer is 50% efficient, generally less than 10 nanometers) absorb light emitted by the QD. Preferred quenchers include commercially available ones sold under the trademark “Black Hole Quencher” but others may be used.

Quantum dots are semiconducting nanocrystals that emit one or more discrete frequencies of light when stimulated by a light source. QDs offer great advantages over conventional organic dyes, including (1) narrow symmetric emission spectra, (2) the ability to use a single light source for the simultaneous excitation of semiconductor QDs with different emission spectra having longer wavelengths than the source, (3) the ability to function through repeated cycles of excitation and fluorescence lasting many hours, and (4) the extreme stability of coated QDs against photobleaching and against changes in the pH of biological electrolytes, which are ubiquitous in biological environments.

In addition, the emission frequencies can be controlled by altering the size of the nanocrystals. By changing reaction conditions, such as the pH of the solution, during the synthesis of the nanocrystals, it is possible to design and produce QDs tailored to emit specific frequencies. These unique optical properties render QDs ideal fluorophores for ultrasensitive and multiplexing applications such as cell labeling and biomolecular detection. A variety of QDs are available commercially.

While a variety of semiconductor materials may be used, it is preferred to use a QD comprising Group II-VI semiconductor material. It is particularly preferred to use a QD having a CdSe—ZnS core-shell that has been functionalized with an organic layer to render it aqueous-compatible. The organic layer may be, but is not limited to, dihydrolipoic acid.

Initially, the QD-antibody complexes are conjugated with the inactivated antigen-quencher complexes. The proximity of the quenchers to the QDs inhibits fluorescence from the QDs via FRET. When a test sample containing unlabeled pathogens (without bound quenchers) is added to the assay solution, equilibrium reactions cause them to displace a fraction of the quencher labeled inactivated target antigens from the QD labeled antibodies. The QDs no longer adjacent to a quencher then fluoresce upon excitation by a suitable signal and the presence of a target antigen can be detected through fluorescence imaging of conjugates trapped on the surface of a porous filter. Fluorescence imaging measurements give an extremely small background signal due to the filtering out of any unconjugated QDs and dissociated QD complexes in the assay solution, and hence results in a more accurate qualitative detection and allows more accurate measurement of antigen concentrations with no danger of false positives. Such a detection scheme avoids the need for simulants for each class of antigens and also greatly reduces the amount of reagents needed for eventual use in a device.

The invention provides an assay test solution for detecting one or more pathogens through the use of FRET including at least one QD bound to an antibody conjugated with at least one inactivated target antigen bound to at least one quencher molecule as described above. Preferably, the QD comprises a Group II-VI semiconductor core. More preferably, the QD includes a CdSe—ZnS core-shell nanocrystal.

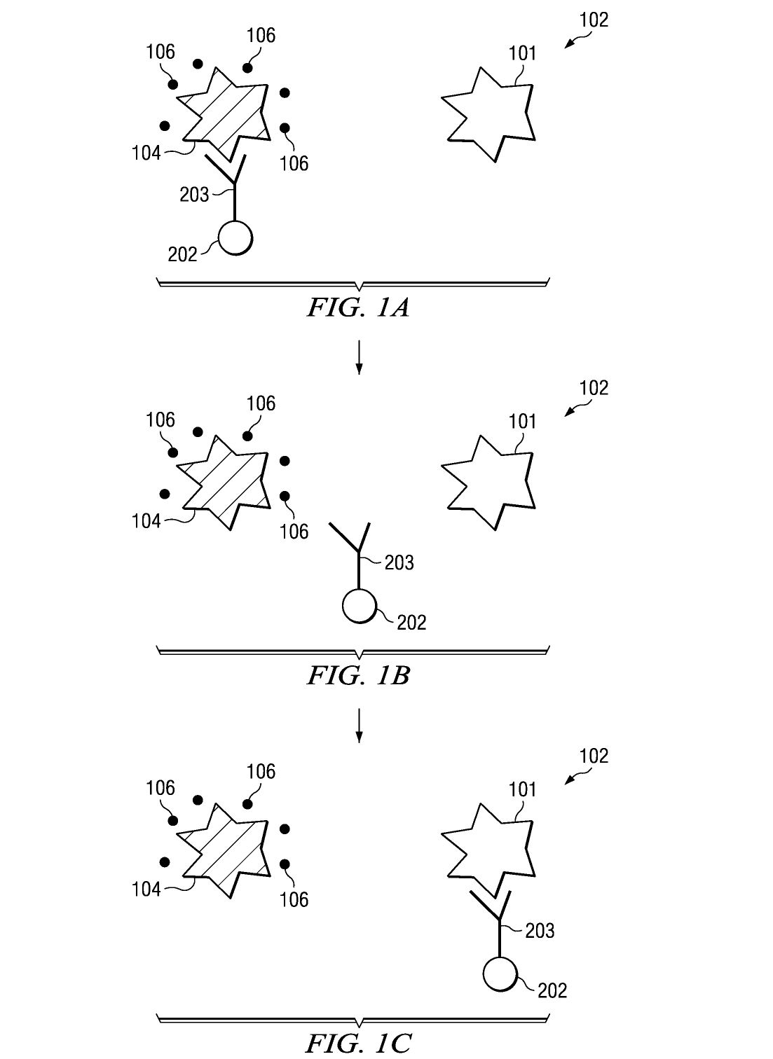

FIGS. 1A-1C show the steps in a dissociation/association process according to the invention. Referring first to FIG. 1A, a complex 102 according to the invention includes the following elements. A functionalized QD 202 is bound to an antibody 203 which has receptor site(s) with which it binds to an inactivated antigen 104. The inactivated antigen 104 previously has had bound to it one or more quencher molecules 106. While the QD 202 will fluoresce upon being illuminated with light of a predetermined wavelength, the fluorescence will be undetectable because any emission from the QD 202 will be absorbed by the quencher molecules 106, which are within the Frster radius of the QD 202. A pathogen 101 will at this point not be associated with the complex 102.

In FIG. 1B, the inactivated antigen 104 has become dissociated from the antibody 203. The quencher molecules 106 bound to the antigen 104 therefore also become dissociated from the antibody 203, and the QD 202 bound to it. The quencher molecules 106 will therefore no longer inhibit the detection of fluorescence from the QD 202.

In FIG. 1C, the QD/antibody complex 202, 203 has become bound to the pathogen 101. This dissociation/reassociation permits the QD fluorophore to tag a pathogen while masking out the fluorescence of any QDs 202 still associated with inactivated antigens 104. An improvement in the signal to noise ratio of the detection system results.

Examples of how to make and use the complexes are described below.

### Example 1

### Antibody/QD Attachment

We first describe the preparation of the assay test solution used in the detection of the biological contaminants. For this study, E. coli 0157:H7 monoclonal antibodies (purchased from Biocompare Inc.) were conjugated to 605 nm QDs, B. cereus antibodies (purchased from Research Diagnostics Inc.) were conjugated to 565 nm QDs, and MS-2 virus antibodies (purchased from Tetracore Inc.) were conjugated to 525 nm QDs. All of the QDs used were CdSe/ZnS carboxyl coated Evitags™ (purchased from Evident Technologies, NY).

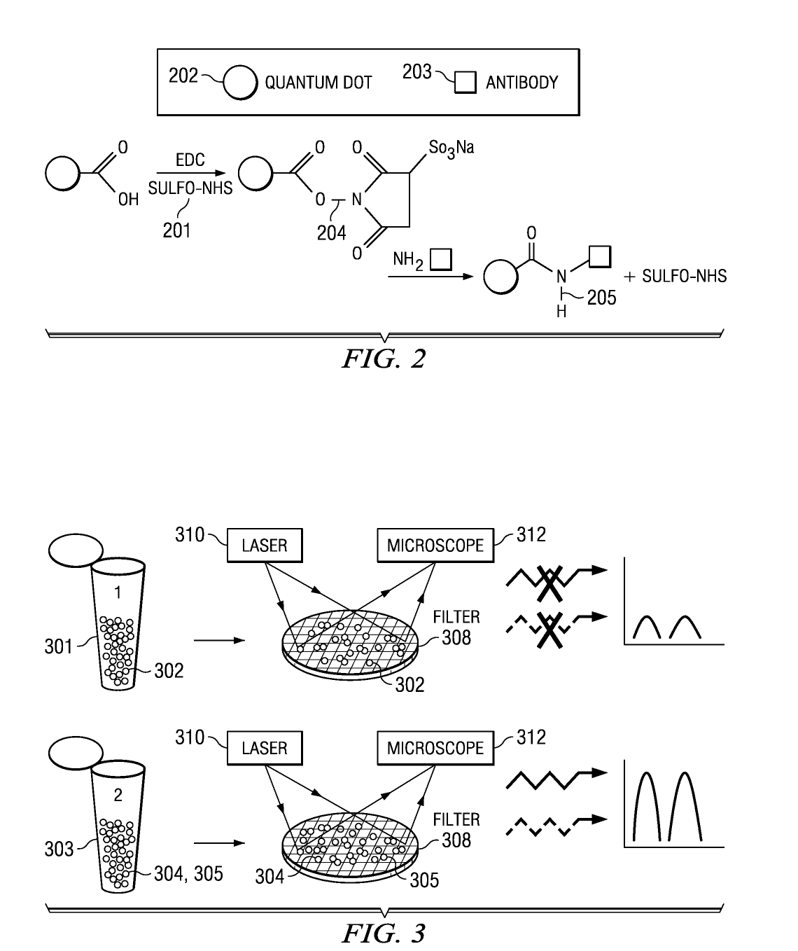

FIG. 2 shows the cross-linking reagents 201 used in functionalizing the QDs 202. Reagents 201 contain reactive ends for specific functional groups (amine and carboxyl groups). Cross-linking agents useful for binding biomolecules to QDs include EDC (1-ethyl-3-(3-dimethylaminopropyl) carbodiimide hydrochloride) and sulfo-NHS(N-hydroxysuccinimide). In the case of QDs functionalized with carboxyl groups, EDC reacts with the carboxylic acid group and activates the carboxyl group to form an active O-acylisourea intermediate, allowing it to be coupled to the amino group in the antibodies. An EDC byproduct is released as a soluble urea derivative after displacement by the nucleophile. The O-acylisourea intermediate is unstable in aqueous solutions, making it ineffective in two-step conjugation procedures unless its stability is increased using N-hydroxysuccinimide. This intermediate reacts with a primary amine to form an amide derivative. The detailed protocol involves the addition of 0.5 mg EDC, 0.375 mg sulfo-NHS, 0.02 ml MES buffer (0.1 M MES, pH 6.0) and 0.03 ml DI water to 50 microliters of a 10 micro molar solution of carboxyl coated QDs in a standard 1.5 ml eppendorf tube. This solution is mixed in a vortex mixer for 15 seconds three times and allowed to stand for 30 minutes in the dark. EDC acts as a dehydrating agent by removing a molecule of water from the carboxylic group and from the hydroxyl group of the Sulfo-NHS. This results in the formation of an amine reactive intermediate, which is stabilized by the presence of Sulfo-NHS. EDC helps form active amine reactive esters on the surface of the QDs. Upon further reaction with a primary amine (an antibody in this case), the QD-antibody conjugate 205 is formed through an amide bond (CONH).

Through this procedure, antibodies for all three different antigens were conjugated to the three different sizes of QDs. However, as was discovered through subsequent photoluminescence and fluorescence measurements, either many of the QDs remained unconjugated or many of the QD/antibody-antigen/quencher complexes became dissociated even in the absence of unlabeled pathogens. These QDs contributed a substantial photoluminescence background in the liquid, but after the liquid was passed through the filters on which the QD-antibody-antigen complexes were trapped for fluorescence imaging, an almost zero background was obtained from the fluorescence imaging.

### Example 2

### Inactivated Target Antigen/Quencher Attachment

Using similar procedures, carboxyl terminated quenchers were conjugated to inactivated E. coli 0157:H7 (purchased from KPL Inc. of Gaithersburg, Md.), inactivated B. cereus (purchased from ATCC Inc. of Manassas, Va.) and MS-2 antigens (obtained from CERL, Champaign, Ill.). Stock solutions were prepared containing 106 colony forming units per milliliter (CFUs/mL) of each type of antigen in standard buffers, and were stored separately according to the manufacturer's specifications before the conjugation reactions. The quencher chosen for the experiment was purchased from Biosearch Technologies of Novato Calif. Specifically the Black Hole Quencher-2 (BHQ-2). BHQ-2 was chosen for its strong absorbance (quenching) over a wide range of wavelengths in the visible region, making it suitable for multiplexing, in which QDs having different emission wavelengths are used for detection. Black Hole Quenchers are organic quencher compounds that offer exceptional quenching with no native fluorescence. BHQ-2 carboxylic acid can be activated and coupled to the amine groups of the antigens.

Briefly, the procedure for the conjugation of quenchers to the antigens involved reacting carboxyl terminated BHQ-2 quenchers with antigens in the presence of EDC (1-ethyl-3-(3-dimethylaminopropyl) carbodiimide hydrochloride) and sulfo-NHS(N-hydroxysuccinimide).

The detailed protocol involves the addition of 0.5 mg EDC, 0.375 mg sulfo-NHS, 0.02 ml MES buffer (0.1 M MES, pH 6.0) and 0.03 ml DI water to 50 μl of a 10 μM solution of carboxyl terminated BHQ-2 in an eppendorf tube. This solution is mixed in a vortex mixer for 15 seconds three times and allowed to stand for 30 minutes in the dark. The carboxyl group intermediate reacts with the primary amine present in the antigen to form an amide bond. BHQ-2 quenchers were conjugated to all three types of antigens. This solution containing the antigen-quencher complexes was then resuspended in 75 μl of a 1× phosphate buffer saline (PBS) buffer and added to 75 μL of corresponding solution of QD-antibody conjugates prepared earlier and was reacted for 15 minutes.

For multiplexed detection all three QD-antibody solutions were reacted with a solution containing a mixture of all three antigen-BHQ-2 complexes. A solution containing free (unlabeled) inactivated antigens was added to the solution containing the complexes and reacted for 2 minutes for concentrations of 10⁵ CFUs/ml and for 5 minutes for concentrations of 10² CFUs/ml. The reaction times were determined based on several experiments (reacting over different time ranges) to determine the minimum time required for noise-free detection at a given concentration.

Before introducing the free inactive antigens into the assay test solution, 0.5 mL of the assay test solution was placed in a quartz cuvette and excited with a 441.6 nm helium cadmium laser to perform photoluminescence measurements. Following this, free inactive antigens were added to the solution to replace some of the quencher labeled antigens in the assay solution, and photoluminescence measurements were taken again. The photoluminescence setup had an incident beam from the laser which was focused on the sample in a quartz cuvette to a spot size of approximately 2 mm in height and 50 μm in width. The intensity of the light was on the order of 65 W/cm². The scattered light from the sample was collected and imaged onto the entrance slit of a SPEX 1877 TripleMate spectrometer equipped with a UV-enhanced liquid-nitrogen-cooled charge coupled device (CCD). High-reflectance dielectric mirrors were used to steer the laser beam and a single plane convex lens, of UV grade fused silica was used to focus the scattered beam. Win Spec™ software was used for data collection and analysis. Due to the presence of unlabelled QDs in the assay solution, a large photoluminescence background signal was obtained even before the replacement of the quencher labeled antigens in the complexes by free inactive antigens. FIG. 3 depicts an illustration of the filtering mechanism used for the FRET-based multiplexing scheme. Vial 1 301 contains only quenched QD complexes 302; vial 2 303 contains quenched and unquenched QD complexes 304, 305. Quenched complexes 302 are trapped on a filter 308 and are illuminated with a laser source 310 selected to have emission wavelengths which will excite the QDs associated with quenched complexes 302. A microscope 312 is used to view the field as illuminated. However, because almost all of the QDs 302 not remaining associated with quenchers, passed through the filter, light emitted from the complexes 302 on the filter is quenched, and low signal strengths result. In the case of the unquenched complexes 304, 305, light emitted from the complexes trapped on filter 308 does not get quenched, resulting in significantly more pronounced emission spectra.

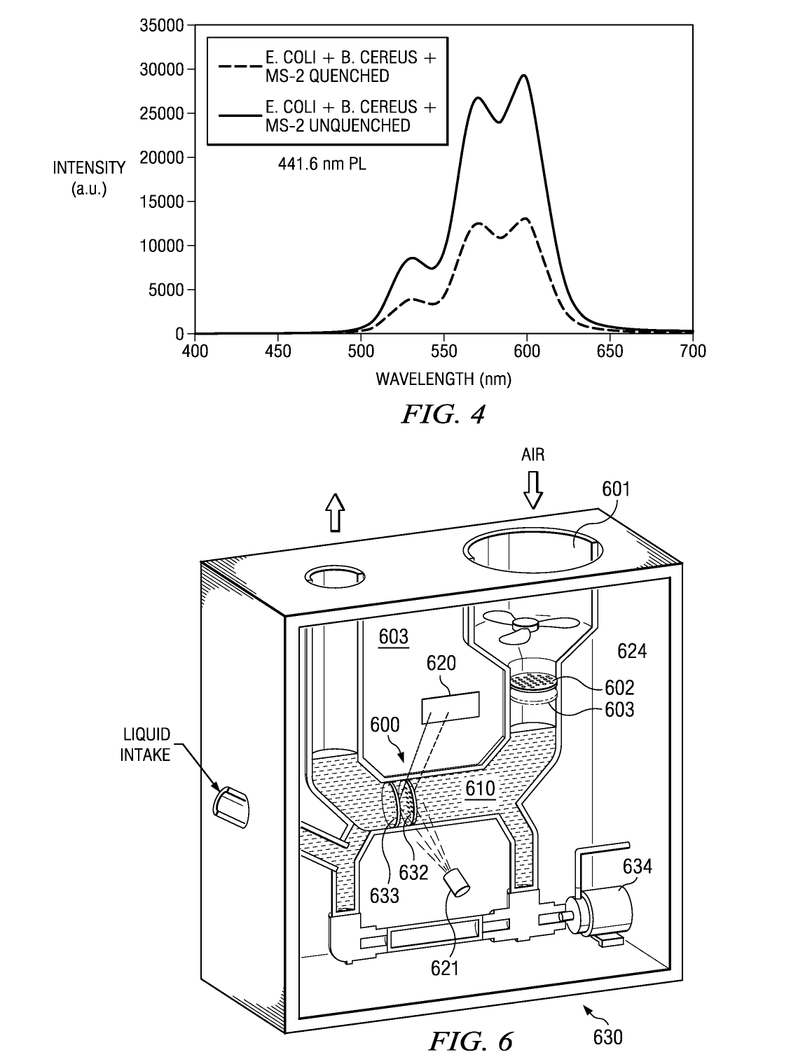

The results from the photoluminescence measurements for the simultaneous detection of all three types of biological antigens are shown in FIG. 4. There are emission peaks associated with each kind of pathogen.

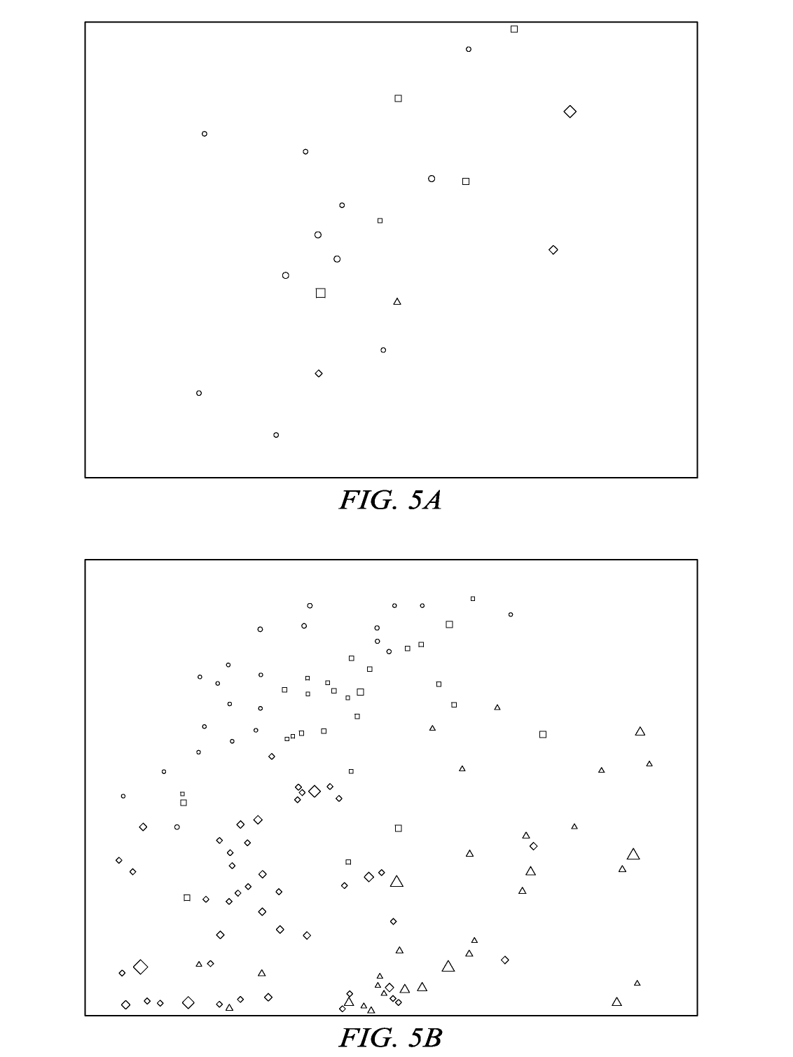

In order to eliminate the background signal due to unlabeled QDs in the solution, these solutions were passed through a 0.2 μm pore size nontortuous filter (purchased from Millipore Inc.) to trap the QD-antigen complexes while allowing the free QDs to pass through the filters. Then, fluorescence imaging was performed on the surface of the filters using a Nikon E600 fluorescence microscope mounted with a CCD. Special band-pass filters (Chroma Technology Corp.) were used to image the fluorescence from the QDs over a narrow detection window (40 nm). The large background due to unlabelled QDs seen in photoluminescence measurements was eliminated because the unlabelled QDs had passed through the Millipore filter trapping just the QDs bound to complexes. The result from the fluorescence imaging for the simultaneous detection of all three types of BAs is shown in FIGS. 5(a) and (b). By employing the filtering scheme almost all the noise in the signal was eliminated and the resulting detection based on this image was devoid of any false-positives.

The inventors have provided a method for detecting pathogens by formulating an assay test solution containing a QD/antibody-antigen/quencher complex made with the procedures described above. A volume of fluid is introduced into a vessel or vessels containing the assay test solution to form a test sample. The method of introduction may be any known to one of skill in the art, including but not limited to bubbling, injection, or flowing. The volume of fluid may be any defined volume or a continuous stream taken from a fluid that is to be tested and which may or may not contain chemical or biological agents to be detected. Chemical agents include regulated and unregulated industrial chemicals, toxins, and any other chemical that provokes an immune system response. Biological agents include, but are not limited to, single or multicellular bacteria, fungi, viruses, algae, spores, or microbes. While no specific size limitation is intended, the invention has been shown to be capable of detecting small (approximately 25 nanometers for the MS2-virus), medium (approximately 1 micron for Bacillus cereus), and large (approximately 3 microns for Escherichia coli) biological agents. Larger pathogens, 10 microns for example, are possible as well.

In a preferred embodiment, the assay test solution includes at least a second QD/antibody-antigen/quencher complex in which the complex comprises an inactivated target antigen different from the inactivated target antigen of the first QD/antibody-antigen/quencher complex.

More preferably, the step of formulating the assay test solution comprises including a second QD/antibody-antigen/quencher complex having a second QD which emits a second wavelength different from the first wavelength and a second antigen which is different from the first antigen. This can be accomplished by specifying that the second QD be of a different size than the first QD. Third, fourth, fifth, etc. QD/antibody-antigen quencher complexes can be added to distinctly detect further pathogens, to the limit of a photodetector's ability to discriminate between the different wavelengths emitted by the QDs.

The test sample may be mixed by any means known in the art, including but not limited to, suspended magnetic particles. The test sample is the held for a predetermined period of time to permit a portion of the QD/antibody-antigen/quencher complexes to dissociate and bind with the pathogens from the volume of fluid to form QD/antibody-pathogen complexes. This predetermined period of time may vary according to the minimum concentration of pathogens desired to be detected. Nonlimiting examples include 2 minutes for concentrations greater than or equal to 10⁵ CFUs/mL and 5 minutes for concentrations greater than or equal to 10² CFUs/mL.

The test sample then passes through one or more detection filters that trap the QD/antibody-antigen/quencher complexes and the QD/antibody-pathogen complexes while the remainder of the fluid passes through. The pore size of the detection filters may vary, but the detection filters preferably capture particles greater than or equal to approximately 25 nanometers, and lets through particles at least as small as the employed, functionalized, but nonbound QDs and QD-antibody fragments. A series of two of more detection filters may be employed to detect larger antigens and pathogens on the first filter and smaller antigens and pathogens on the following filters. An optional particle filter may be included upstream of the detection filter to trap large particles, but must allow particles smaller than approximately 5 microns to pass through.

A light source (or sources) then illuminates the detection filter(s), causing the QD on each quantum dot/antibody-pathogen complex to emit a particular frequency of light. In some embodiments, the light source may be a broad spectrum light source capable of exciting many different types of fluorescent dyes or QDs and, in other embodiments, the light source may emit one or more narrow frequencies of light and may include such light sources as light emitting diodes (hereinafter LEDs) among others. A photodetector detects the light from the QD/antibody-pathogen complexes, indicating the presence of the pathogen.

Representative apparatus 630 is shown in FIG. 6. Apparatus 630 may be a part of an HVAC system. An intake 601 intakes fluid which then passes through at least one dust filter 602 that traps particles between about ten to about twenty microns in size that spans an intake conduit 624. Optionally, a second dust filter 603 with pore sizes larger or smaller than dust filter 602. A means for drawing in a volume of the air or other fluid may be a fan, blower, compressor, gravity, or partial vacuum.

Conduit 624 leads to a vessel 600 in which the introduced fluid is tested, as by mixing it with or bubbling the fluid through a test solution 610. It is contemplated that there may be one or more vessels 600 containing an assay test solution 610 so that the cycle time between sample readings may be reduced. The vessel or vessels 600 containing the assay test solution may have positive, negative, or atmospheric pressures and may be made of any material including metal, plastic, polymer, and/or transparent materials such as glass. The primary requirement is that a photodetector 620, preferably positioned alongside a transparent section of a vessel 600, be able to sense the light emitted from the QDs. Additionally, the vessel may be of any size or shape including cylinders, spheres, tubes, cubes, or channels, and may be simply a continuation of the conduit 624.

The assay test fluid 610 comprises the QD/antibody-antigen/quencher complexes discussed above and may contain other additives such as surfactants, biocides, stabilizers, nutrients, thickeners, gels, colloids, coagulants, thinners, or dyes.

The vessel 600 may also be in fluid communication with a reservoir containing additional assay test solution by a valved inlet and in fluid communication with a drain by a valved outlet.

At least one detection filter 632 is positioned within or to form one boundary of the vessel 600, so as to be viewable by photodetectors 620 and illuminable by LEDs 621. The detection filter(s) 632 may be made of any suitable material with nontortuous pore sizes that will trap the QD/antibody-antigen/quencher complexes and the QD/antigen-pathogen complexes, while allowing the remainder of the fluid (including unassociated QDs) to pass through. Such materials include, but are not limited to clear polycarbonate. Additionally, the detection filter 632 may cover a portion of or the entire cross section of the vessel 600 and be of any shape desired and may be positioned anywhere within the vessel, e.g. in the center or near a wall. It is anticipated that the position of the detection filter and prefilter may need to be changed periodically. As such, they could be rotated in and out of the vessel by a filter wheel. Optionally, a second detection filter 633 may be added in and may have pore sizes that are larger or smaller than the first detection filter 632.

The apparatus 630 also includes a means for moving a portion of the test sample through the detection filter. The means for moving may be a pump 634, compressor, blower, or other means and can be implemented through techniques known in the art.

Suitable light sources for illuminating the detection filter include broad spectrum light sources capable of exciting many different types of QDs or, in other embodiments, light sources capable of emitting one or more narrow frequencies of light exciting only specific QDs. Such light sources may include LEDs 621, among others. It is contemplated that a light source capable of serially emitting discrete frequencies may be used to activate QDs in a specific sequence, permitting a time-divided detection of different pathogens rather than a detection protocol that depends only on differences in color.

The photodetector(s) 620 then detect the light from the QD/antibody-pathogen complexes, indicating the presence of the pathogen. Suitable photodetectors include but are not limited to optical detectors, photoresistors, photodiodes, charge-coupled devices (CCDs), and other devices such as are known in the art. Of particular importance is the ability of the photodetector to discriminate between two or more frequencies.

In summary, methods and apparatus for the rapid and simultaneous detection of multiple kinds of pathogens have been disclosed in which QDs are used as labeling fluorophores. QDs associated with inactivated antigens and quencher molecules do not detectably fluoresce, while QDs associated with unquenched pathogens do. The use of QDs instead of fluorescent dyes produces higher signal to noise ratios and makes the complexes more survivable in real-life environments.

While illustrated embodiments of the present invention have been described and illustrated in the appended drawings, the present invention is not limited thereto but only by the scope and spirit of the appended claims.

We claim:

1. A method for detecting one or more pathogens through the use of fluorescent resonance energy transfer, comprising the steps of:
formulating an assay test solution to contain at least a first QD/antibody-inactivated antigen/quencher complex;
introducing a volume of fluid, possibly containing pathogens, into a vessel containing the assay test solution;
mixing the volume of fluid into the assay test solution in the vessel to form a test sample solution;
holding the test sample solution for a predetermined period of time, allowing a portion of the first QD/antibody-inactivated antigen/quencher complexes to dissociate into QD/antibody groups and antigen/quencher groups;
allowing the QD/antibody groups to attach to the one or more pathogens forming QD/antibody-pathogen complexes;
passing the test sample solution through at least one detection filter, wherein the detection filter(s) traps QD/antibody-antigen/quencher complexes and QD/antibody-pathogen complexes while allowing the remainder of the fluid to pass through;
illuminating the detection filter(s) with a light source;
responsive to said step of illuminating, emitting photons having a first wavelength from the QDs on the detection filter(s); and
detecting the light of the first wavelength from QDs on the detection filter(s) not associated with quenchers with a photodetector to indicate the presence of the pathogen.

2. The method of claim 1, wherein the assay test solution includes a second QD/antibody-antigen/quencher complex, the second QD/antibody-antigen/quencher complex comprising an inactivated target antigen different from the inactivated target antigen of the first QD/antibody-antigen/quencher complex.

3. The method of claim 1, wherein the photons emitted from the QDs have two or more wavelengths.

4. The method of claim 1, further comprising the step of passing the volume of fluid through at least one prefilter to trap particles above a predetermined size, but allowing pathogens in the volume of fluid to pass through.

5. The method of claim 4, wherein the predetermined size is in the range of about ten to about twenty microns.

6. The method of claim 1, wherein the step of formulating the assay test solution comprises the step of functionalizing the QDs with at least one cross-linking agent.

7. The method of claim 6, wherein the at least one cross-linking agent is 1-ethyl-3-(3-dimethylaminopropyl) carbodiimide hydrochloride and N-hydroxysuccinimide.

8. The method of claim 1, wherein the step of formulating the assay test solution comprises the step of including a second QD/antibody-antigen/quencher complex having a second QD which emits a second wavelength different from the first wavelength and a second antigen which is different from the first antigen.

9. The method of claim 1, wherein the QD comprises a Group II-VI semiconductor core.

10. The method of claim 9, wherein the QD comprises a CdSe—ZnS core-shell nanocrystal.

11. The method of claim 1, wherein the QD is coated with an organic monolayer to make the QD aqueous-compatible.

12. The method of claim 1, wherein the at least one pathogen is greater than approximately 25 nanometers and less than approximately 10 microns in size.

13. The method of claim 1, wherein the pathogen is selected from the group consisting of Escherichia Coli 0157:H7, Bacillus Cereus, and MS-2.

14. The method of claim 1, wherein the at least one detection filter comprises a plurality of detection filters.

15. The method of claim 14, wherein the plurality of detection filters have different pore sizes.

16. The method of claim 1, wherein the pathogen is any chemical or biological agent, substance, or organism that provokes an immune system to produce an antibody or antibodies.

17. The method of claim 1, wherein the fluid is air or water.

18. The method of claim 1, wherein the predetermined period is 2 minutes and the method has a sensitivity of detecting pathogens at of least 10² CFUs/mL.

19. The method of claim 1, wherein the predetermined period is 5 minutes and the method has a sensitivity of detecting pathogens at of least 10⁵ CFUs/mL.

20. Apparatus for detecting a pathogen through the use of fluorescent resonance energy transfer, comprising:
an intake for drawing in a fluid to be tested for at least one antigen;
means for moving a volume of fluid into the intake;
a vessel of assay test solution, the assay test solution including
a quencher molecule bound to at least one inactivated target antigen conjugated with an antibody bound to at least one QD;
a conduit coupling the intake to the vessel so as to introduce the volume of fluid into the assay test solution to form a test sample;
at least one detection filter in communication with the vessel, wherein the detection filter(s) traps QD/antibody-antigen/quencher complexes and QD/antibody-pathogen complexes but allows the remainder of the fluid to pass through;
means for flowing at least a portion of the test sample solution through the detection filter(s);
a light source illuminating the detection filter(s); and
a photodetector for detecting at least one predetermined wavelength which is emitted by a QD when the quencher is not bound to the complex including the QD.

21. The apparatus of claim 20, wherein the light source is one or more LEDs.

22. The apparatus of claim 20, further comprising a dust filter inside the conduit for removing particles larger than a predetermined size, but allowing pathogens smaller than approximately 10 microns in the volume of fluid to pass through.

23. The apparatus of claim 20, wherein the photodetector discriminates between at least two different wavelengths.

24. The apparatus of claim 20, wherein the means for moving a volume of fluid is a fan, pump, compressor, blower, partial vacuum, or gravity.

25. The apparatus of claim 20, wherein the means for flowing at least a portion of the test sample through the detection filter(s) is a pump, compressor, blower, partial vacuum, or gravity.

26. The apparatus of claim 20, wherein the fluid is air or water.

27. The apparatus of claim 20, wherein the at least one detection filter comprises a plurality of detection filters.

28. The apparatus of claim 20, wherein the plurality of detection filters have different pore sizes.

29. An assay test solution for detecting one or more pathogens through the use of fluorescent resonance energy transfer, comprising:
a quencher molecule bound to at least one inactivated target antigen conjugated with an antibody bound to at least one QD.

30. The solution of claim 29, wherein the QD comprises a Group II-VI semiconductor core.

31. The solution of claim 29, wherein the QD comprises a CdSe—ZnS core-shell nanocrystal.

32. The solution of claim 29, further comprising a second QD/antibody-antigen/quencher complex having a second QD which emits a second wavelength different from the first wavelength and a second antigen which is different from the first antigen.
